(19) United States
(12) Patent Application Publication
(10) Pub. No.: US 2024/0346809 A1
(43) Pub. Date: Oct. 17, 2024

(54) PROCEDURAL VIDEO ASSESSMENT

(71) Applicant: Intel Corporation, Santa Clara, CA (US)

(72) Inventors: Ping GUO, Beijing (CN); Mee Sim LAI, Penang (MY); Kuan Heng LEE, Ayer Itam (MY); Wee Hoo CHEAH, Taipei, Taiwan (CN); Jason GARCIA, Scottsdale, AZ (US); Liang QIU, Shanghai (CN); Peng WANG, Beijing (CN); Jiajie WU, Shanghai (CN); Xiangbin WU, Beijing (CN)

(73) Assignee: Intel Corporation, Santa Clara, CA (US)

(21) Appl. No.: 18/574,935

(22) PCT Filed: Jan. 7, 2022
(86) PCT No.: PCT/CN2022/070828

§ 371 Date: Dec. 28, 2023

(51) Int. Cl. G06V 10/776; G06V 10/70; G06V 10/77; G06V 10/774; G06V 10/82; G06V 20/40; G09B 19/00

(52) U.S. Cl. CPC G06V 10/776; G06V 10/768; G06V 10/7715; G06V 10/774; G06V 20/41; G06V 20/44; G06V 20/49; G09B 19/003; G06V 10/82

(57) ABSTRACT

The application provides an apparatus and a method for procedural video assessment. The apparatus includes: interface circuitry; and processor circuitry coupled to the interface circuitry and configured to: perform an action segmentation process for a procedural video received via the interface circuitry to obtain a plurality of action features associated with the procedure video; transform the plurality of action features into a plurality of action-procedure features based on an action-procedure relationship learning module for discovering a relationship between the plurality of action features and a plurality of scoring oriented procedures associated with the procedure video; and perform a procedure classification process to infer the plurality of scoring oriented procedures from the plurality of action-procedure features.

### TECHNICAL FIELD

Embodiments described herein generally relate to artificial intelligence (AI), and more particularly relate to procedural video assessment.

### BACKGROUND

A procedural video, also known as an instructional video or a how-to video, captures a process of completing a particular task, e.g., cooking, assembling, or conducting a science experiment. Scoring of the procedural video is to evaluate how well a person performs the task at each step. It is important to evaluate people's performance without manual intervention, e.g., to detect an unqualified working process in factory to improve product quality.

### BRIEF DESCRIPTION OF THE DRAWINGS

The various advantages of the embodiments will become apparent to one skilled in the art by reading the following specification and appended claims, and by referencing the following drawings, in which:

FIG. 1 shows example actions, procedures, key frames and scoring items in a balance weighting experiment according to some embodiments of the present disclosure;

FIG. 2A shows a schematic diagram for illustrating a typical method for key frame extraction:

FIG. 2B shows a schematic diagram for illustrating a typical method for procedure segmentation:

FIG. 3 shows a schematic diagram for illustrating a solution for scoring oriented procedure segmentation and key frame extraction according to some embodiments of the present disclosure:

FIG. 4 shows example key frames with scoring as expected outputs of scoring oriented key frame extraction according to some embodiments of the present disclosure:

FIG. 5 shows an example auto-scoring framework based on scoring oriented procedure segmentation and key frame extraction according to some embodiments of the present disclosure:

FIG. 6 shows an example implementation of an auto-scoring solution based on scoring oriented procedure segmentation and key frame extraction according to some embodiments of the present disclosure:

FIG. 7 shows an example flow chart of a method for procedural video assessment according to some embodiments of the present disclosure:

FIG. 8 is a block diagram illustrating components, according to some example embodiments, able to read instructions from a machine-readable or computer-readable medium and perform any one or more of the methodologies discussed herein; and

FIG. 9 is a block diagram of an example processor platform in accordance with some embodiments of the disclosure.

### DETAILED DESCRIPTION

Various aspects of the illustrative embodiments will be described using terms commonly employed by those skilled in the art to convey the substance of the disclosure to others skilled in the art. However, it will be apparent to those skilled in the art that many alternate embodiments may be practiced using portions of the described aspects. For purposes of explanation, specific numbers, materials, and configurations are set forth in order to provide a thorough understanding of the illustrative embodiments. However, it will be apparent to those skilled in the art that alternate embodiments may be practiced without the specific details. In other instances, well-known features may have been omitted or simplified in order to avoid obscuring the illustrative embodiments.

Further, various operations will be described as multiple discrete operations, in turn, in a manner that is most helpful in understanding the illustrative embodiments: however, the order of description should not be construed as to imply that these operations are necessarily order dependent. In particular, these operations need not be performed in the order of presentation.

Procedure assessment is a new trend in recent years. For example, starting from 2023, China MOE (Minister of Education) mandates Science (Physics, Chemistry and Biology) Lab Experiments as part of the entrance test from middle school to high school. Using AI to effectively perform process streamlining, especially solutions like auto-scoring/semi-auto-scoring systems, will be desired due to the large number of students to achieve that.

A procedural video captures a process of completing a particular task, e.g., cooking, assembling, conducting a science experiment. Scoring of a procedural video (i.e. procedural video assessment) is to evaluate how well a person performs the task at each step. It is important to evaluate people's performance without manual intervention, e.g., to detect the unqualified working process in factory to improve product quality.

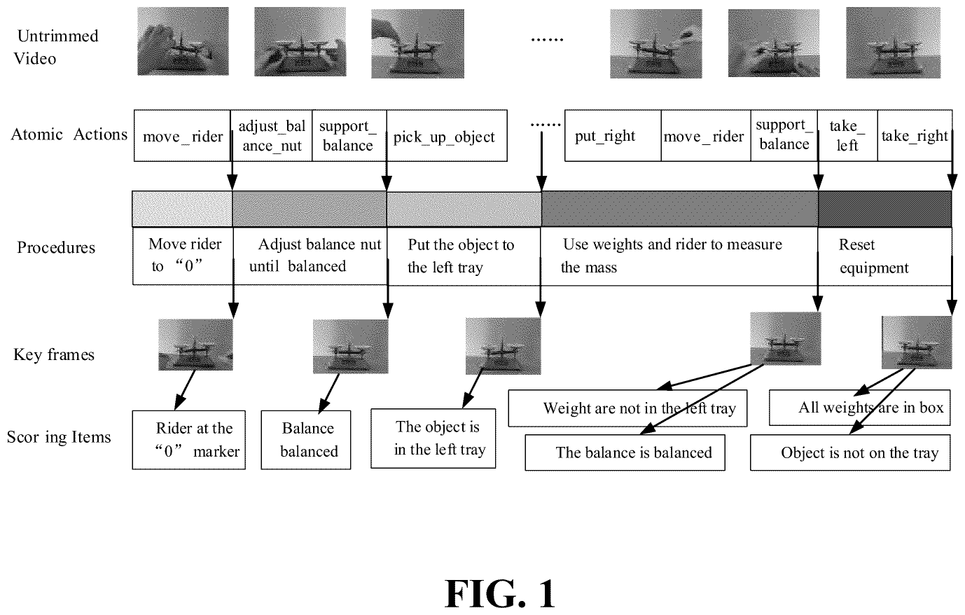

It usually takes multiple procedures with temporal dependencies to finish a task. For example, in a balance weighting experiment as shown in FIG. 1, a student may be asked to complete five procedures: moving a rider of a balance to a “0” marker: adjusting a balance nut to balance a beam: putting an object on a left tray: using weights and the rider to measure a mass of the object; and resetting the equipment. Some procedures may include only one action and some other procedures may include a sequence of actions. Each procedure may be associated with one or several scoring items, and thus the procedure may be referred to as a scoring oriented procedure herein. For each procedure, when the procedure is completed, the student's performance for completing the procedure may be assessed based on the scoring items associated with the procedure and a scoring result for completing the procedure may be generated. In this way, after all the procedures in the experiment are completed, a total scoring result of the student may be generated.

According to the above description, in order to perform auto-scoring based on a procedural video, it is important to segment the procedural video into the procedures associated with the scoring items, and then key frames for the procedures may be extracted for auto-scoring.

As shown in FIG. 1, an input video is an untrimmed procedural video and key frames for scoring oriented procedures need to be extracted for scoring purpose. According to embodiments of the present disclosure, a solution for scoring oriented procedure segmentation and key frame extraction is proposed to extract key frames accurately for procedural scoring. Generally, the procedural video may be firstly segmented into atomic actions, then the actions may be classified into respective scoring oriented procedures based on action-procedure relationship learning so as to segment the procedure video into scoring oriented procedures, and then key frames associated with the scoring oriented procedures may be extracted for auto-scoring based on predefined scoring items. For example, as shown in FIG. 1, the ending frame of each procedure is extracted as the key frame associated with the procedure. However, it is easily understood that the key frame associated with the procedure may be not limited to the ending frame of the procedure, but may be any appropriate frame in the procedure depending on actual applications.

It is noted that in addition to the application shown in FIG. 1, the proposed solution can also be used in many other applications, for example, to assess manual procedures of a manufacturing technician in industry, to assess if an exercise is done correctly in healthcare, and to assess a worker's ergonomics at their workstation for office fitness, etc. It is also noted that the proposed solution is currently illustrated based on a single video source, but it can be extended to multiple video sources. For example, in an implementation on a smart science lab project, two views for capturing the procedures of the project may be used and then results for the two views can be combined.

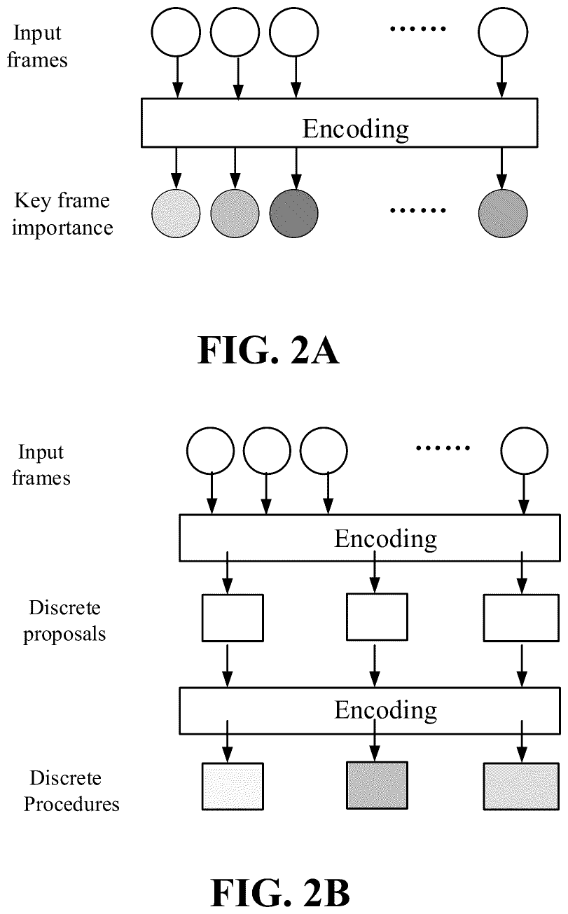

In the embodiments of the disclosure, based on the scoring oriented procedure segmentation, the key frames may be extracted accurately for procedural scoring. In contrast, existing methods for key frame extraction or procedure segmentation mainly focus on clustering of frames based on their similarity, detection of discrete actions, and scoring each individual action. FIG. 2A shows a schematic diagram for illustrating a typical method for key frame extraction, and FIG. 2B shows a schematic diagram for illustrating a typical method for procedure segmentation.

Specifically, FIG. 2A shows a key frame extraction method described in Z. Ji, K. Xiong, Y. Pang and X. Li, “Video Summarization With Attention-Based Encoder-Decoder Networks,” in IEEE Transactions on Circuits and Systems for Video Technology, vol. 30, no. 6, pp. 1709-1717 June 2020, doi: 10.1109/TCSVT.2019.2904996, which extracts key frames from untrimmed videos by learning an importance weight of each frame. FIG. 2B shows a procedure segmentation method described in Zhou, Luowei, Chenliang Xu, and Jason J. Corso. “Towards automatic learning of procedures from web instructional videos.” Thirty-Second AAAI Conference on Artificial Intelligence. 2018, which utilizes an action detection framework to first generate procedure proposals and then classifies each proposal with a label.

As illustrated, existing methods for key frame extraction or procedure segmentation focus on how to segment actions and learns the importance of each frame directly. However, these methods are not designed for scoring purpose and not optimal in extracting key frames for scoring, and thus suffer from low accuracies for scoring applications.

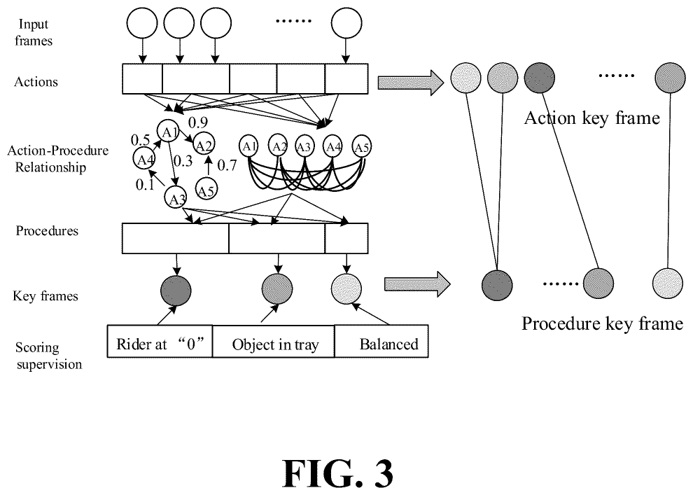

According to the present disclosure, a solution for scoring oriented procedure segmentation and key frame extraction is proposed. FIG. 3 shows a schematic diagram for illustrating a solution for scoring oriented procedure segmentation and key frame extraction according to some embodiments of the present disclosure. As shown in FIG. 3, the proposed solution may include an action-procedure relationship learning module and a scoring supervision module to improve the key frame extraction for procedural scoring purpose. By use of the proposed solution, hierarchical key frames with action-procedure relations may be generated as outputs to enable scoring for procedural video assessment.

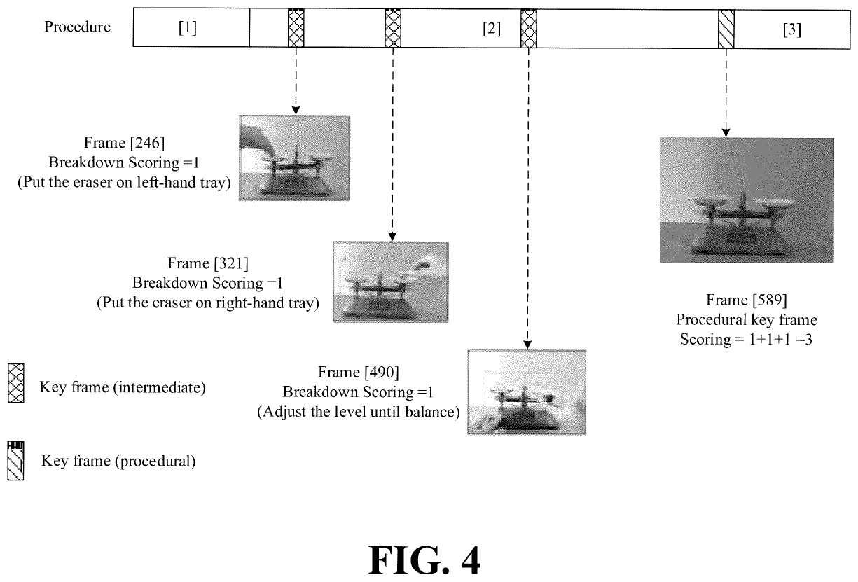

FIG. 4 shows example key frames with scoring as expected outputs of scoring oriented key frame extraction according to some embodiments of the present disclosure. As shown in FIG. 4, for example, the task may include three procedures. For each procedure, a procedural key frame (also referred to as a main key frame herein) may be output with a final scoring for indicating a user's performance of completing the procedure, and one or several intermediate key frames may be also output with respective breakdown scorings. The intermediate key frames may show important actions or objects and corresponding breakdown scorings prior to the procedural key frame. In an example, for the procedure [2] shown in FIG. 4, Frame at the ending of the procedure may be extracted as the procedural key frame and output with the final scoring 3, and three intermediate key frames (Frame [246], Frame and Frame [490]) may be also extracted and output with respective breakdown scorings. It is noted that the main key frame associated with the procedure may be not limited to the ending frame of the procedure, but may be any appropriate frame in the procedure depending on actual applications.

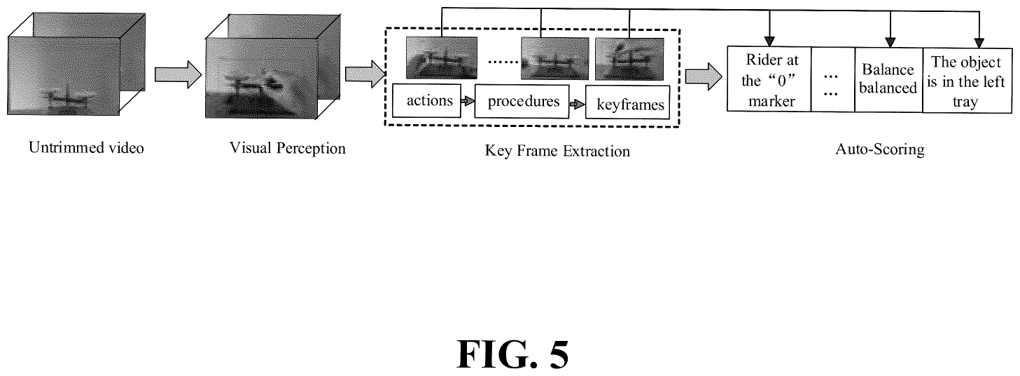

In the present disclosure, the proposed solution for enabling auto-scoring based on scoring oriented procedure segmentation and key frame extraction will be further described in detail with reference to FIG. 5 to FIG. 7.

FIG. 5 shows an example auto-scoring framework based on scoring oriented procedure segmentation and key frame extraction according to some embodiments of the present disclosure. According to the illustration of FIG. 5, given an input untrimmed video, a perception module may perform visual perception on the video, which may include object detection, hand detection, face recognition, emotion recognition, etc.; a key frame extraction module may extract key frames on which the scoring is conducted; and an auto-scoring module may perform auto-scoring on each extracted key frame to give a score on each scoring items associated with the key frame. In the key frame extraction module, the above described solution for scoring oriented procedure segmentation and key frame extraction may be used.

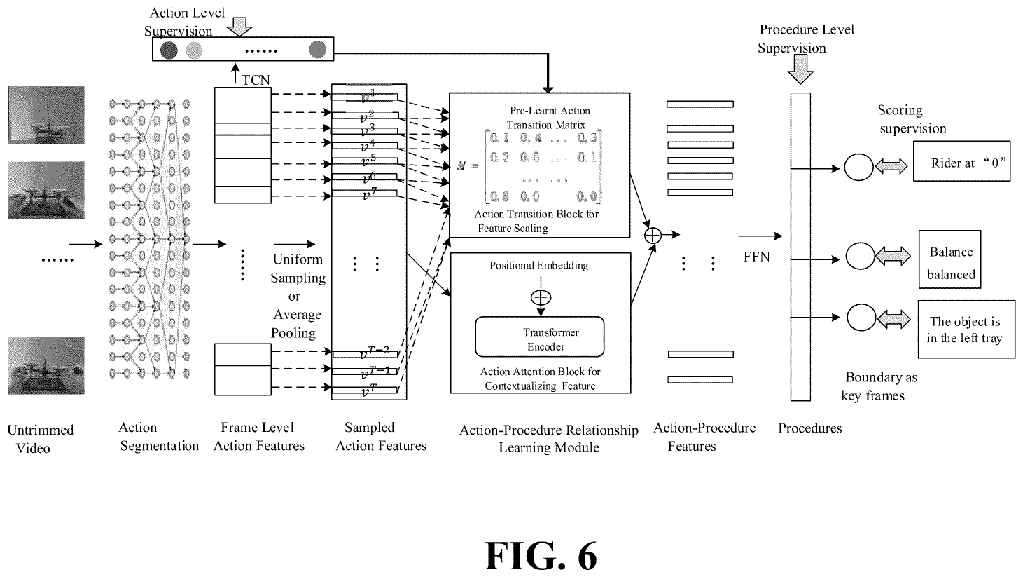

Based on the above described overall framework for auto-scoring, FIG. 6 shows an example implementation of an auto-scoring solution based on scoring oriented procedure segmentation and key frame extraction according to some embodiments of the present disclosure.

According to the illustration of FIG. 6, given an untrimmed procedural video as input, an action segmentation process may be performed for the procedural video to obtain a plurality of action features associated with the procedural video. Any existing or future neural network module for action segmentation may be used to perform the action segmentation process, which is not limited herein. For example, the improved Multi-Stage Temporal Convolutional Network (MS-TCN++) described in Shi-Jie Li, Yazan AbuFarha, Yun Liu, Ming-Ming Cheng, and Juergen Gall, “MS-TCN++: Multi-stage temporal convolutional network for action segmentation. TPAMI, 2020” may be used to segment the procedural video into the plurality of frame level action features. Then, the frame level action features may be uniformly sampled in the temporal dimension to reduce the computational cost in the following process modules. After the uniform sampling, the sampled action features may be obtained. Alternatively, an average pooling may be performed to get the sampled action features.

The sampled action features may be fed into an action-procedure relationship learning module for transforming the action features into action-procedure features. The action-procedure features may imply information about the action-procedure relationship learnt in the action-procedure relationship learning module. The action-procedure features may be fed into a feed forward network (FFN) to achieve procedure classification so as to obtain scoring oriented procedures. The boundary frames of the scoring oriented procedures may be extracted as key frames, and then an auto-scoring algorithm may be conducted on the key frames to give scores corresponding to the key frames.

In some embodiments of the disclosure, as shown in FIG. 6, the action-procedure relationship learning module may include an action attention block for contextualizing the action features based on action attentions learnt for the action features. The action attention block may be used to learn full range contexts in the temporal dimension. Specifically, in the action attention block, self and mutual importance of each action feature and positional embedding information for each action feature may be learnt and used for contextualizing the action features so as to transform the action features into the action-procedure features. For example, a temporal transformer encoder described in Sharir, Gilad, Asaf Noy, and Lihi Zelnik-Manor. “An Image is Worth 16×16 Words, What is a Video Worth?” arXiv preprint arXiv: 2103.13915 (2021) may be adopted in the action attention block to learn the full range attention at action level and transform the action features into the action-procedure features.

In some embodiments of the disclosure, the action-procedure relationship learning module may further include an action transition block for scaling the action features based on a pre-learnt action transition matrix M. For example, a labeled dataset may be used to learn an action transition matrix M where each element indicates the transition probability from one action to another. The matrix M may have a size of A×A where A is the number of action types. The sampled action features may be denoted as vectors v^{t}, t=1 . . . . T where T is the sequence length (number of vectors) after sampling. The action prediction label of v^{t} may be denoted as a^{t}, t∈{1, . . . A}. The action feature vector v^{t} may be updated by the action transition block as follows:

$$\\ \begin{matrix}{f}_{tran} ( {v}^{{}_{} t} ) = 0.5 \cdot ( M ( {a}^{{}_{} t - 1} , {a}^{{}_{} t} ) + M ( {a}^{{}_{} t} , {a}^{{}_{} t + 1} ) ) {v}^{{}_{} t} . & ( 2 )\end{matrix}$$

The action transition block may be applied to scale the action features by domain knowledge. For example, in the balance weighting experiment, the action “put weights to the right tray” unlikely happens after the action “take the object from left tray”. By multiplying the action feature vector with a scaling factor as defined by the above equation (2), the response of low confident actions can be reduced based on the domain knowledge.

Thus when the action-procedure relationship learning module include both the action attention block and the action transition block, the outputs from the two blocks may be fused as the action-procedure features.

According to embodiments of the disclosure, the proposed auto-scoring solution may be implemented in a neural network, and three levels of supervision may be utilized for training various layers in the neural network. As shown in FIG. 6, the three levels of supervision may include: i) an action level supervision that labels each frame with an action type associated with the frame; ii) a procedure level supervision that labels each frame with a procedure type associated with the frame; and iii) a scoring oriented supervision that labels each frame with a score on each scoring item associated with the frame.

For the scoring oriented supervision, suppose there are K procedures predicted in the neural network and K key frames are extracted by using the ending frame of each procedure. Then the association between key frames and scoring items may be built. For example, in FIG. 1, the scoring item “Rider at the “0” marker” should be scored on the key frame after the procedure of “Move rider to the ‘0’ marker”. The consistent loss function for auto-scoring may be defined as follows:

$$\\ \begin{matrix}g = {\sum }_{k = 1}^{K} {( AG ( {f}_{k} ) - {G}_{k} )}^{2} & ( 1 )\end{matrix}$$

where Gₖ is the ground truth score for the k-th procedure, fₖ is the key frame at the k-th procedure, and AG (.) is the auto-scoring function. In an example implementation, expert defined rules may be used as the AG (.).

The action level supervision and the procedure level supervision may be applied for training of related layers in the neural network. For example, the action segmentation process may be trained based on the action level supervision, the procedure classification process may be trained based on the procedure level supervision. Also, the action transition matrix used in the action transition block may be trained based on the action level supervision.

In some embodiments of the disclosure, based on the above-described three levels of supervision, the final loss function applied for training may be defined as follows:

$$\\ \begin{matrix}L = \frac{1}{N} {\sum }_{n = 1}^{N} - log ( {y}_{n , a} ) + \frac{1}{T} {\sum }_{t = 1}^{T} - log ( {y}_{t , p} ) + α \cdot g & ( 3 )\end{matrix}$$

where N is the number of input frames, y_{n,a} is the predicted probability for the ground truth action label α at the n-th frame, Tis the number of sampled action features, y_{t,p} is the predicted probability for the ground truth procedure label p at the t-th sampled action feature, a is a weighted parameter and g is the scoring consistency constraint defined in Equation (1).

To sum up, a solution for scoring oriented procedure segmentation and key frame extraction is proposed in the disclosure to extract key frames accurately for procedural scoring. The comparison between the proposed solution and existing solutions (e.g. as shown in FIG. 2A and FIG. 2B) is shown in Table 1 below. As shown in Table 1, according to the proposed solution, the training function may need inputs of multi-level ground truth labels, and the inference function may output both action and procedure segments.

TABLE 1
Comparison of the proposed solution with existing solutions

| | Existing solutions | Proposed solution |
|---|---|---|
| Data | frame-level action | frame-level action AND |
| Annotation | OR/AND key frame | procedure (duration of frames), |
| required for | per procedure | AND scoring per procedure |
| Training | | |
| Inference | single-level key | hierarchical frame(s) AND the |
| outputs | frame(s) | relevant score |

In order to compare the performance of the proposed solution with the existing solution, an example system for the “balance weighting” experiment is set up where two views of videos are used for action segmentation, procedure segmentation and key frame extraction. In this example system, 8 action types and 3 procedure types are defined. The dataset for training contains 27 pairs of videos by 7 performers under two views, in which 14 pairs of videos are used for training and 13 pairs of videos are used for testing. As an example, the proposed solution is compared with the solution shown in FIG. 2A. The end to end performance is evaluated by calculating the accuracy of the final auto-scoring result. With the proposed solution, the accuracy is significantly improved from 58% to 77%.

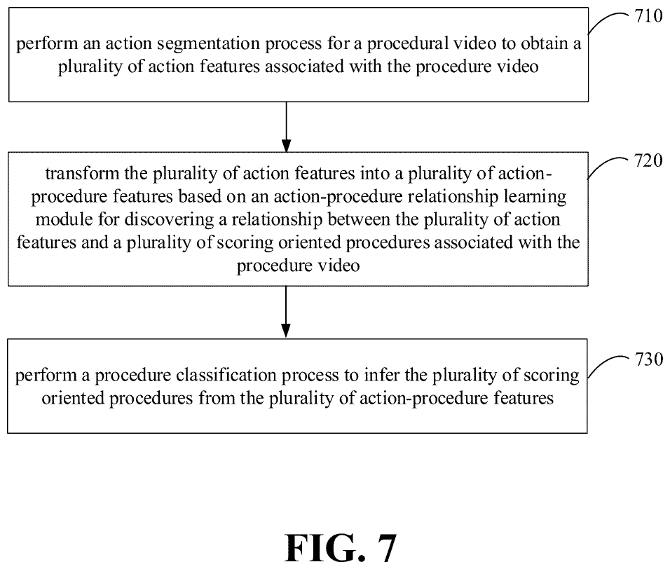

FIG. 7 shows an example flow chart of a method for procedural video assessment according to some embodiments of the present disclosure. The method may be implemented by a processor and include operations 710 to 730.

At operation 710, the processor may perform an action segmentation process for a procedural video to obtain a plurality of action features associated with the procedure video.

In some embodiments, the processor may further perform uniform sampling or average pooling in a temporal dimension after the action segmentation process to obtain sampled action features in the temporal dimension as the plurality of action features.

At operation 720, the processor may transform the plurality of action features into a plurality of action-procedure features based on an action-procedure relationship learning module for discovering a relationship between the plurality of action features and a plurality of scoring oriented procedures associated with the procedure video.

In some embodiments, the action-procedure relationship learning module may include an action attention block for contextualizing the action features based on action attentions learnt for the action features.

In some embodiments, the action-procedure relationship learning module may further include an action transition block for scaling the action features based on a pre-learnt action transition matrix.

At operation 730, the processor may perform a procedure classification process to infer the plurality of scoring oriented procedures from the plurality of action-procedure features.

In some embodiments, the action segmentation process may be trained based on an action level supervision that labels each frame with an action type associated with the frame.

In some embodiments, the procedure classification process may be trained based on a procedure level supervision that labels each frame with a procedure type associated with the frame.

In some embodiments, the processor may further perform a key frame extraction process to extract, for each scoring oriented procedure, a main key frame to show completeness of the procedure.

In some embodiments, the processor may further perform the key frame extraction process to extract, for each scoring oriented procedure, one or more intermediate key frames to show one or more important actions or objects in the procedure.

In some embodiments, the main key frame may be an ending frame of the procedure.

In some embodiments, the processor may further perform auto-scoring for the main key frame of each scoring oriented procedure by use of an auto-scoring algorithm and based on one or more predetermined scoring items associated with the procedure.

In some embodiments, the key frame extraction process may be trained based on a scoring oriented supervision that labels each frame with a score on each scoring item associated with the frame.

In some embodiments, the processor may further perform a visual perception on the procedural video before the action segmentation process. The visual perception may include object detection, hand detection, face recognition, or emotion recognition.

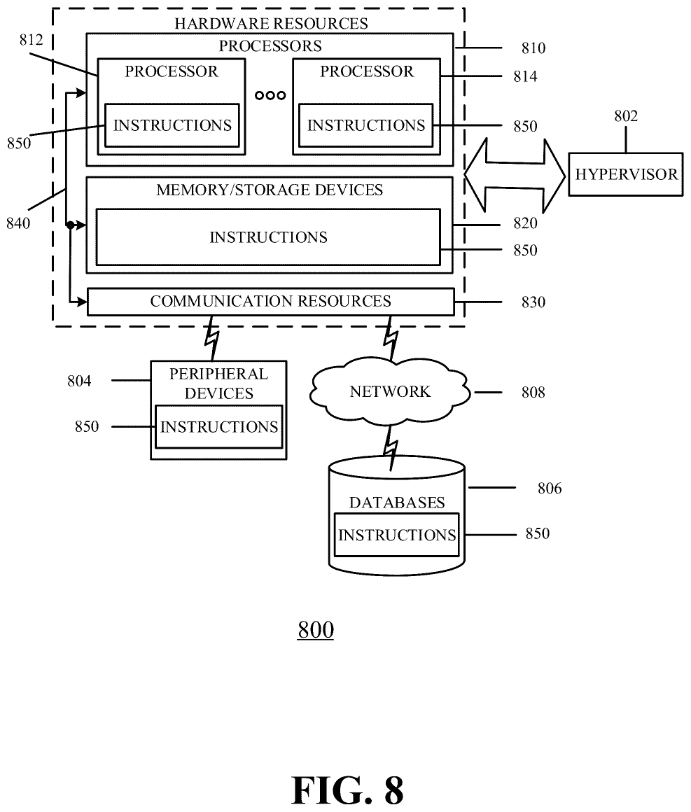

FIG. 8 is a block diagram illustrating components, according to some example embodiments, able to read instructions from a machine-readable or computer-readable medium (e.g., a non-transitory machine-readable storage medium) and perform any one or more of the methodologies discussed herein. Specifically, FIG. 8 shows a diagrammatic representation of hardware resources 800 including one or more processors (or processor cores) 810, one or more memory/storage devices 820, and one or more communication resources 830, each of which may be communicatively coupled via a bus 840. For embodiments where node virtualization (e.g., NFV) is utilized, a hypervisor 802 may be executed to provide an execution environment for one or more network slices/sub-slices to utilize the hardware resources 800.

The processors 810 may include, for example, a processor 812 and a processor 814 which may be, e.g., a central processing unit (CPU), a graphics processing unit (GPU), a tensor processing unit (TPU), a visual processing unit (VPU), a field programmable gate array (FPGA), or any suitable combination thereof.

The memory/storage devices 820 may include main memory, disk storage, or any suitable combination thereof. The memory/storage devices 820 may include, but are not limited to any type of volatile or non-volatile memory such as dynamic random access memory (DRAM), static random-access memory (SRAM), erasable programmable read-only memory (EPROM), electrically erasable programmable read-only memory (EEPROM), Flash memory, solid-state storage, etc.

The communication resources 830 may include interconnection or network interface components or other suitable devices to communicate with one or more peripheral devices 804 or one or more databases 806 via a network 808. For example, the communication resources 830 may include wired communication components (e.g., for coupling via a Universal Serial Bus (USB)), cellular communication components, NFC components, Bluetooth® components (e.g., Bluetooth® Low Energy), Wi-Fi® components, and other communication components.

Instructions 850 may comprise software, a program, an application, an applet, an app, or other executable code for causing at least any of the processors 810 to perform any one or more of the methodologies discussed herein. The instructions 850 may reside, completely or partially, within at least one of the processors 810 (e.g., within the processor's cache memory), the memory/storage devices 820, or any suitable combination thereof. Furthermore, any portion of the instructions 850 may be transferred to the hardware resources 800 from any combination of the peripheral devices 804 or the databases 806. Accordingly, the memory of processors 810, the memory/storage devices 820, the peripheral devices 804, and the databases 806 are examples of computer-readable and machine-readable media.

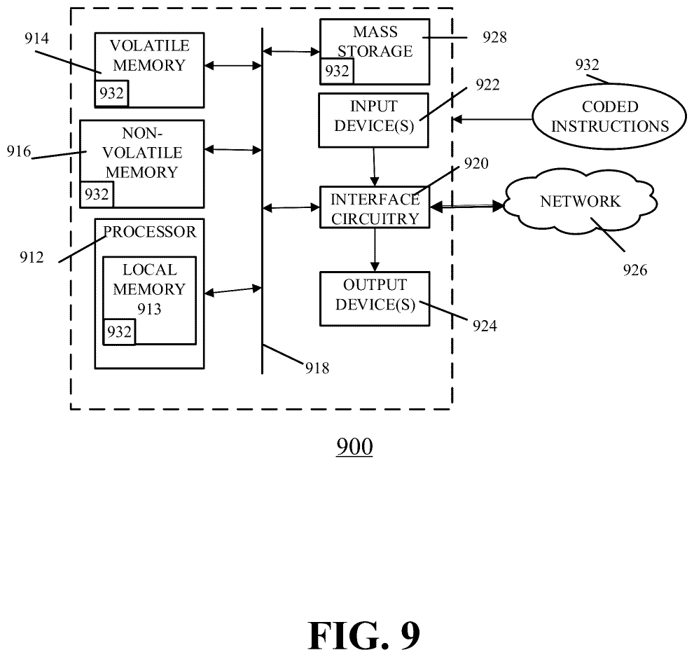

FIG. 9 is a block diagram of an example processor platform in accordance with some embodiments of the disclosure. The processor platform 900 can be, for example, a server, a personal computer, a workstation, a self-learning machine (e.g., a neural network), a mobile device (e.g., a cell phone, a smart phone, a tablet such as an iPad™), a personal digital assistant (PDA), an Internet appliance, a DVD player, a CD player, a digital video recorder, a Blu-ray player, a gaming console, a personal video recorder, a set top box, a headset or other wearable device, or any other type of computing device.

The processor platform 900 of the illustrated example includes a processor 912. The processor 912 of the illustrated example is hardware. For example, the processor 912 can be implemented by one or more integrated circuits, logic circuits, microprocessors, GPUs, DSPs, or controllers from any desired family or manufacturer. The hardware processor may be a semiconductor based (e.g., silicon based) device. In some embodiments, the processor implements one or more of the methods or processes described above.

The processor 912 of the illustrated example includes a local memory 913 (e.g., a cache). The processor 912 of the illustrated example is in communication with a main memory including a volatile memory 914 and a non-volatile memory 916 via a bus 918. The volatile memory 914 may be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 916 may be implemented by flash memory and/or any other desired type of memory device. Access to the main memory 914, 916 is controlled by a memory controller.

The processor platform 900 of the illustrated example also includes interface circuitry 920. The interface circuitry 920 may be implemented by any type of interface standard, such as an Ethernet interface, a universal serial bus (USB), a Bluetooth® interface, a near field communication (NFC) interface, and/or a PCI express interface.

In the illustrated example, one or more input devices 922 are connected to the interface circuitry 920. The input device(s) 922 permit(s) a user to enter data and/or commands into the processor 912. The input device(s) can be implemented by, for example, an audio sensor, a microphone, a camera (still or video), a keyboard, a button, a mouse, a touchscreen, a track-pad, a trackball, and/or a voice recognition system.

One or more output devices 924 are also connected to the interface circuitry 920 of the illustrated example. The output devices 924 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuitry 920 of the illustrated example, thus, typically includes a graphics driver card, a graphics driver chip and/or a graphics driver processor.

The interface circuitry 920 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 926. The communication can be via, for example, an Ethernet connection, a digital subscriber line (DSL) connection, a telephone line connection, a coaxial cable system, a satellite system, a line-of-site wireless system, a cellular telephone system, etc.

For example, the interface circuitry 920 may include a training dataset inputted through the input device(s) 922 or retrieved from the network 926.

The processor platform 900 of the illustrated example also includes one or more mass storage devices 928 for storing software and/or data. Examples of such mass storage devices 928 include floppy disk drives, hard drive disks, compact disk drives, Blu-ray disk drives, redundant array of independent disks (RAID) systems, and digital versatile disk (DVD) drives.

Machine executable instructions 932 may be stored in the mass storage device 928, in the volatile memory 914, in the non-volatile memory 916, and/or on a removable non-transitory computer readable storage medium such as a CD or DVD.

### Additional Notes and Examples

Example 1 includes an apparatus for procedural video assessment, comprising: interface circuitry; and processor circuitry coupled to the interface circuitry and configured to: perform an action segmentation process for a procedural video received via the interface circuitry to obtain a plurality of action features associated with the procedure video: transform the plurality of action features into a plurality of action-procedure features based on an action-procedure relationship learning module for discovering a relationship between the plurality of action features and a plurality of scoring oriented procedures associated with the procedure video; and perform a procedure classification process to infer the plurality of scoring oriented procedures from the plurality of action-procedure features.

Example 2 includes the apparatus of Example 1, wherein the processor circuitry is further configured to: perform a key frame extraction process to extract, for each scoring oriented procedure, a main key frame to show completeness of the procedure.

Example 3 includes the apparatus of Example 2, wherein the processor circuitry is further configured to: perform the key frame extraction process to extract, for each scoring oriented procedure, one or more intermediate key frames to show one or more important actions or objects in the procedure.

Example 4 includes the apparatus of Example 2, wherein the main key frame is an ending frame of the procedure.

Example 5 includes the apparatus of Example 2, wherein the processor circuitry is further configured to: perform auto-scoring for the main key frame of each scoring oriented procedure by use of an auto-scoring algorithm and based on one or more predetermined scoring items associated with the procedure.

Example 6 includes the apparatus of Example 5, wherein the key frame extraction process is trained based on a scoring oriented supervision that labels each frame with a score on each scoring item associated with the frame.

Example 7 includes the apparatus of any of Examples 1 to 6, wherein the action-procedure relationship learning module comprises an action attention block for contextualizing the action features based on action attentions learnt for the action features.

Example 8 includes the apparatus of Example 7, wherein the action-procedure relationship learning module further comprises an action transition block for scaling the action features based on a pre-learnt action transition matrix.

Example 9 includes the apparatus of any of Examples 1 to 6, wherein the processor circuitry is further configured to perform uniform sampling or average pooling in a temporal dimension after the action segmentation process to obtain sampled action features in the temporal dimension as the plurality of action features.

Example 10 includes the apparatus of any of Examples 1 to 6, wherein the processor circuitry is further configured to perform a visual perception on the procedural video before the action segmentation process.

Example 11 includes the apparatus of Example 10, wherein the visual perception comprises at least one of object detection, hand detection, face recognition, or emotion recognition.

Example 12 includes the apparatus of any of Examples 1 to 6, wherein the action segmentation process is trained based on an action level supervision that labels each frame with an action type associated with the frame.

Example 13 includes the apparatus of any of Examples 1 to 6, wherein the procedure classification process is trained based on a procedure level supervision that labels each frame with a procedure type associated with the frame.

Example 14 includes a method for procedural video assessment, comprising: performing an action segmentation process for a procedural video to obtain a plurality of action features associated with the procedure video; transforming the plurality of action features into a plurality of action-procedure features based on an action-procedure relationship learning module for discovering a relationship between the plurality of action features and a plurality of scoring oriented procedures associated with the procedure video; and performing a procedure classification process to infer the plurality of scoring oriented procedures from the plurality of action-procedure features.

Example 15 includes the method of Example 14, further comprising: performing a key frame extraction process to extract, for each scoring oriented procedure, a main key frame to show completeness of the procedure.

Example 16 includes the method of Example 15, further comprising: performing the key frame extraction process to extract, for each scoring oriented procedure, one or more intermediate key frames to show one or more important actions or objects in the procedure.

Example 17 includes the method of Example 15, wherein the main key frame is an ending frame of the procedure.

Example 18 includes the method of Example 15, further comprising: performing auto-scoring for the main key frame of each scoring oriented procedure by use of an auto-scoring algorithm and based on one or more predetermined scoring items associated with the procedure.

Example 19 includes the method of Example 18, wherein the key frame extraction process is trained based on a scoring oriented supervision that labels each frame with a score on each scoring item associated with the frame.

Example 20 includes the method of any of Examples 14 to 19, wherein the action-procedure relationship learning module comprises an action attention block for contextualizing the action features based on action attentions learnt for the action features.

Example 21 includes the method of Example 20, wherein the action-procedure relationship learning module further comprises an action transition block for scaling the action features based on a pre-learnt action transition matrix.

Example 22 includes the method of any of Examples 14 to 19, further comprising: performing uniform sampling or average pooling in a temporal dimension after the action segmentation process to obtain sampled action features in the temporal dimension as the plurality of action features.

Example 23 includes the method of any of Examples 14 to 19, further comprising: performing a visual perception on the procedural video before the action segmentation process.

Example 24 includes the method of Example 23, wherein the visual perception comprises at least one of object detection, hand detection, face recognition, or emotion recognition.

Example 25 includes the method of any of Examples 14 to 19, wherein the action segmentation process is trained based on an action level supervision that labels each frame with an action type associated with the frame.

Example 26 includes the method of any of Examples 14 to 19, wherein the procedure classification process is trained based on a procedure level supervision that labels each frame with a procedure type associated with the frame.

Example 27 includes a computer-readable medium having instructions stored thereon, wherein the instructions, when executed by processor circuitry, cause the processor circuitry to perform the method of any of Examples 14 to 26.

Example 28 includes an apparatus for procedural video assessment, comprising means for performing the method of any of Examples 14 to 26.

Various techniques, or certain aspects or portions thereof, may take the form of program code (i.e., instructions) embodied in tangible media, such as floppy diskettes, CD-ROMs, hard drives, non-transitory computer readable storage medium, or any other machine-readable storage medium, wherein, when the program code is loaded into and executed by a machine, such as a computer, the machine becomes an apparatus for practicing the various techniques. The non-transitory computer readable storage medium may be a computer readable storage medium that does not include signal. In the case of program code execution on programmable computers, the computing system may include a processor, a storage medium readable by the processor (including volatile and non-volatile memory and/or storage elements), at least one input device, and at least one output device. The volatile and non-volatile memory and/or storage elements may be a RAM, EPROM, flash drive, optical drive, magnetic hard drive, solid state drive, or other medium for storing electronic data. One or more programs that may implement or utilize the various techniques described herein may use an application programming interface (API), reusable controls, and the like. Such programs may be implemented in a high level procedural or object oriented programming language to communicate with a computer system. However, the program(s) may be implemented in assembly or machine language, if desired. In any case, the language may be a compiled or interpreted language, and combined with hardware implementations. Exemplary systems or devices may include without limitation, laptop computers, tablet computers, desktop computers, smart phones, computer terminals and servers, storage databases, and other electronics which utilize circuitry and programmable memory, such as household appliances, smart televisions, digital video disc (DVD) players, heating, ventilating, and air conditioning (HVAC) controllers, light switches, and the like.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document: for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

1-25. (canceled)

26. An apparatus, comprising:
interface circuitry; and
processor circuitry coupled to the interface circuitry and configured to:
perform an action segmentation process for a procedural video received via the interface circuitry to obtain a plurality of action features associated with the procedure video;
transform the plurality of action features into a plurality of action-procedure features based on an action-procedure relationship learning module for discovering a relationship between the plurality of action features and a plurality of scoring oriented procedures associated with the procedure video; and
perform a procedure classification process to infer the plurality of scoring oriented procedures from the plurality of action-procedure features.

27. The apparatus of claim 26, wherein the processor circuitry is further configured to: perform a key frame extraction process to extract, for each scoring oriented procedure, a main key frame to show completeness of the procedure.

28. The apparatus of claim 27, wherein the processor circuitry is further configured to: perform the key frame extraction process to extract, for each scoring oriented procedure, one or more intermediate key frames to show one or more important actions or objects in the procedure.

29. The apparatus of claim 27, wherein the main key frame is an ending frame of the procedure.

30. The apparatus of claim 27, wherein the processor circuitry is further configured to: perform auto-scoring for the main key frame of each scoring oriented procedure by use of an auto-scoring algorithm and based on one or more predetermined scoring items associated with the procedure.

31. The apparatus of claim 30, wherein the key frame extraction process is trained based on a scoring oriented supervision that labels each frame with a score on each scoring item associated with the frame.

32. The apparatus of claim 26, wherein the action-procedure relationship learning module comprises an action attention block for contextualizing the action features based on action attentions learnt for the action features.

33. The apparatus of claim 32, wherein the action-procedure relationship learning module further comprises an action transition block for scaling the action features based on a pre-learnt action transition matrix.

34. The apparatus of claim 26, wherein the processor circuitry is further configured to perform uniform sampling or average pooling in a temporal dimension after the action segmentation process to obtain sampled action features in the temporal dimension as the plurality of action features.

35. The apparatus of claim 26, wherein the processor circuitry is further configured to perform a visual perception on the procedural video before the action segmentation process.

36. The apparatus of claim 35, wherein the visual perception comprises at least one of object detection, hand detection, face recognition, or emotion recognition.

37. The apparatus of claim 26, wherein the action segmentation process is trained based on an action level supervision that labels each frame with an action type associated with the frame.

38. The apparatus of claim 26, wherein the procedure classification process is trained based on a procedure level supervision that labels each frame with a procedure type associated with the frame.

39. A method, comprising:
performing an action segmentation process for a procedural video to obtain a plurality of action features associated with the procedure video;
transforming the plurality of action features into a plurality of action-procedure features based on an action-procedure relationship learning module for discovering a relationship between the plurality of action features and a plurality of scoring oriented procedures associated with the procedure video; and
performing a procedure classification process to infer the plurality of scoring oriented procedures from the plurality of action-procedure features.

40. The method of claim 39, further comprising: performing a key frame extraction process to extract, for each scoring oriented procedure, a main key frame to show completeness of the procedure.

41. The method of claim 40, further comprising: performing the key frame extraction process to extract, for each scoring oriented procedure, one or more intermediate key frames to show one or more important actions or objects in the procedure.

42. The method of claim 40, wherein the main key frame is an ending frame of the procedure.

43. The method of claim 40, further comprising: performing auto-scoring for the main key frame of each scoring oriented procedure by use of an auto-scoring algorithm and based on one or more predetermined scoring items associated with the procedure.

44. The method of claim 43, wherein the key frame extraction process is trained based on a scoring oriented supervision that labels each frame with a score on each scoring item associated with the frame.

45. The method of claim 39, wherein the action-procedure relationship learning module comprises an action attention block for contextualizing the action features based on action attentions learnt for the action features.

46. The method of claim 45, wherein the action-procedure relationship learning module further comprises an action transition block for scaling the action features based on a pre-learnt action transition matrix.

47. The method of claim 39, further comprising: performing uniform sampling or average pooling in a temporal dimension after the action segmentation process to obtain sampled action features in the temporal dimension as the plurality of action features.

48. The method of claim 39, further comprising: performing a visual perception on the procedural video before the action segmentation process.

49. The method of claim 48, wherein the visual perception comprises at least one of object detection, hand detection, face recognition, or emotion recognition.

50. A non-transitory computer-readable medium having instructions stored thereon, wherein the instructions, when executed by processor circuitry, cause the processor circuitry to perform the method of claim 39.
